(19) United States
(12) Patent Application Publication
(10) Pub. No.: US 2021/0038912 A1
(43) Pub. Date: Feb. 11, 2021

(54) ADIPOSE TISSUE REDUCTION AND MUSCLE TONING DEVICE

(71) Applicant: Mark Julian, New Port Richey, FL (US)

(72) Inventor: Mark Julian, New Port Richey, FL (US)

(21) Appl. No.: 16/532,481

(22) Filed: Aug. 6, 2019

(51) Int. Cl. A61N 5/06; A61N 1/36

(52) U.S. Cl. CPC A61N 5/0625; A61N 1/3603; A61N 2005/005; A61N 2005/0652; A61N 2005/0626

(57) ABSTRACT

A lipolysis apparatus that has a low power light source and an Electronic Muscle Stimulator that is used on a patient to remove adipose tissue while toning the person's muscles. The light source is in a range of red light with wavelength ranging from 620 nm to 629 nm and 10 MW to 50 MW in power output. The pulse rate of the Electronic Muscle Stimulator is between 1 Hz to 500 Hz.

### REFERENCE TO RELATED APPLICATIONS

The present application is a continuation in part application and claims the benefit of U.S. patent application Ser. No. 15/681,340, which was titled APPARATUS AND METHOD FOR ADIPOSE TISSUE REDUCTION that was filed on Aug. 19, 2017, that claimed the benefit of Provisional Patent Application No. 62/494,003 filed on Aug. 22, 2016, and Provisional Patent Application No. 62/764,442, which was titled EMS WITH LED LIPO that was filed on Aug. 6, 2018, which are incorporated herein by reference.

### BACKGROUND OF THE INVENTION

### Field of the Invention

The present invention is in the technical field of medical devices, and more particularly, to non-invasive medical devices for fat removal, body shaping, and body contouring. The non-invasive device of the present invention uses low-power laser irradiation, LED light waves, and electrical muscle stimulation “EMS” to remove fat while stimulating muscle growth.

### DESCRIPTION OF RELATED ART

There is a great demand for removal of excess adipose tissue, or fat, for health and aesthetic reasons. Cosmetic surgical procedure known as liposuction has been applied widely for targeted and local fat removal, body shaping, and body contouring. In a typical liposuction procedure, excess adipose tissue is suctioned from the body of a patient through one or more Incisions. Since liposuction is an invasive surgical procedure, serious complications including deep vein thrombosis, organ perforation, bleeding, and infection may occur. The post-procedure recovery period is long and often accompanied by a great deal of inflammation, bruising and pain.

Non-invasive methods using electromagnetic energy, such as microwave, ultrasound or radio-frequency radiation, have been applied in fat reduction and body shaping. In particular, procedures and apparatuses have been developed to use low-power laser for non-invasive fat removal or reduction. For example, as described in U.S. Pat. No. 6,605,079B2, Neira, et al. applied one or more treatments of 635 nm laser energy externally to the patient to release at least a portion of the intracellular fat into the interstitial space through a transitory pore in the fat cell. Upon sufficient doses of this low level laser energy at 635 nm, the cell membrane is believed to be momentarily disrupted, releasing the intracellular fat into the interstitial space. Upon cessation of the energy application, the pore closes and the cell membrane returns to contiguity. The released fat is expected to be removed from the patient's body through one or more of the patient's normal bodily systems, including the lymphatic system.

Several non-invasive low-power laser systems have been developed in recent years to perform fat reduction and body contouring utilizing similar physical mechanism as mentioned above, including Zerona by Erchonia Corp, Strawberry by Laser Lipo Ltd, I-Lipo by Chromogenex, and a few others. These systems are being used commercially and have met commercial success in various degrees. However, the efficacy of this type of systems may be limited by the amount of fat released from cells undergoing membrane disruption due to laser irradiation, as well as by the amount of fat removal through normal bodily systems including the lymphatic system. An object of the present Invention is to provide a non-invasive method of low-level laser therapy that increases the efficacy from removing fat from a patient.

The present invention is an improvement of the Inventor's prior Invention wherein the adipose tissue reduction device of his prior invention now incorporates an Electronic Muscle Stimulator so that the areas targeted for adipose tissue reduction are electronically stimulated to develop muscle tone in the areas targeted

### SUMMARY OF THE INVENTION

The present invention is a non-invasive lipolysis device that uses a low power laser, low powered light source an LED light source to irradiate skin and an electronic muscle stimulator “EMS” to develop muscle tone in the irradiated skin areas. The efficacy of low-level laser of the device is enhanced by applying an electrical muscle stimulation simultaneously to the areas irradiated. The electronic muscle stimulation accelerates cellular fat release and lymphatic drainage, and therefore works simultaneously with the low-level light wave irradiation to remove fat while developing muscle tone in the irradiated areas

The present invention uses a low power laser, a low powered light source, or an LED red light wave in the range of 620 nm to 660 nm. More particularly, it uses a beam of red light which may or may not be condensed through a lens or transparent window to obtain the same effect as lipolysis of adipose tissue exposed through incision using a conventional ultrasonic or low power laser. In addition, the lipolysis device includes a flexible head that can adhere to the body and vibration motors inside head. When the unit is used it is adhered to the body the light wave emits and vibration waves emit at the same time which readily discharges liquefied fat from an adipose cell and concentrated in a space between cell tissues through the groin area, where lymphatic vessels are abundant, and out of the body. Further the lipolysis device can stably contact human skin to break down fat and thus is convenient to use. Furthermore, the lipolysis device can break down subcutaneous fat by irradiating an abdominal region with a low power red light wave either through laser or LED light waves without skin damage or surgical operation, thereby effectively removing abdominal fat. Generally, an apparatus for generating a low power laser or LED includes a diode for emitting a red beam light wave having an output of 10 MW to 48 MW and a wavelength that is between 620 nm to 629 nm, and a low power diode driver for arbitrarily adjusting the amount of beam emitted from the diode. Various apparatuses for obtaining a curative effect by irradiating spots on the body with a low power laser suitable for acupuncture or infected parts of the body. However, since the red light wave requires a separate radiation structure, there is no way of breaking down fat by non-invasively irradiating a human body. Meanwhile, in order to effectively treat obesity using a laser light wave, Neira, et al. discloses a new suction lipectomy capable of liquefying fat during suction lipectomy using a low power laser.

The EMS of the present invention delivers a pulse rate in the range from I to 500 Hz.

It is another aspect of the present invention to provide a belt, and so on, when the beam is disposed in the soft materials in an array, a red light wave capable of being output appropriately to non-invasively break down fat.

### BRIEF DESCRIPTION OF THE DRAWINGS

These and other features, aspects, and advantages of the present invention will become better understood with regards to the following description, appended claims, and drawings where:

FIG. 1 is a diagram illustrating the main components of an embodiment of the lipolysis apparatus;

FIG. 2 is a diagram illustrating the main electronic components of the lipolysis apparatus;

FIG. 3 (a) is a perspective front view of an embodiment of the lipolysis apparatus;

FIG. 3 (b) is a perspective back view of an embodiment of the lipolysis apparatus;

FIG. 4 is a perspective view of the lipolysis apparatus when “folded”,

FIG. 5 is a photograph of an alternative embodiment of the lipolysis apparatus that shows its dual control unit;

FIG. 6 is a photograph of the alternative embodiment of the lipolysis apparatus that shows its low power light source and its Electronic Muscle Stimulator;

FIG. 7 is a photograph of alternative embodiment of the lipolysis apparatus that shows its low power light source;

FIG. 8 is a photograph of an alternative embodiment of the lipolysis apparatus that shows its Electric Muscle Stimulators placed over the low power light sources; and

FIG. 9 is a photograph of an alternative embodiment of the lipolysis apparatus that shows its dual control unit

### REFERENCE NUMERALS IN THE DRAWINGS

For a more complete understanding of the present invention parts, reference is now made to the following descriptions:
100 Control unit.
102 Operator input.
103 Display panel.
104 Timer.
200 Treatment applicator.
201 Power inlet.
202 PCB control board.
203 Solid state relays.
204 Light source.
205 Vibration motor.
206 Treatment panel.
207 Motor housing.
208 Transparent window.
209 Applicator frame.
210 strap loop.
211 Flexible contact frame.
212 strap.
300 Connection cable.

### DETAILED DESCRIPTION OF THE INVENTION

The following detailed description includes preferred embodiments of the invention. The description is not to be taken in a limiting sense, but is made merely for the purpose of illustrating the general principles of the invention.

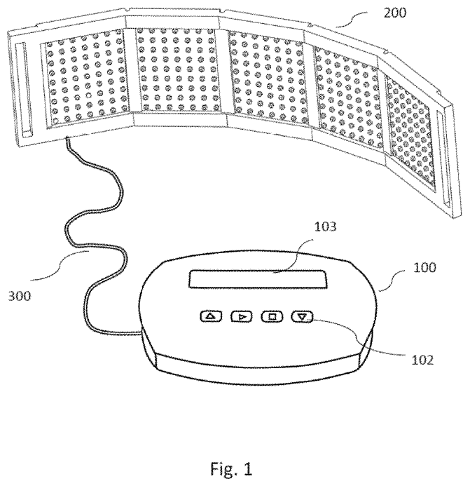

FIG. 1 is a diagram illustrating the main components of an embodiment of a lipolysis apparatus using a low power laser, a low powered light source or an LED light source. The portable apparatus comprises a control unit (100), a treatment applicator (200), and a connection cable (300) connecting the control unit (100) and the treatment applicator (200). On the control unit (100), there is an operator interface comprising input buttons (102) for entering setting parameters and a display panel (103) for displaying system and timer status.

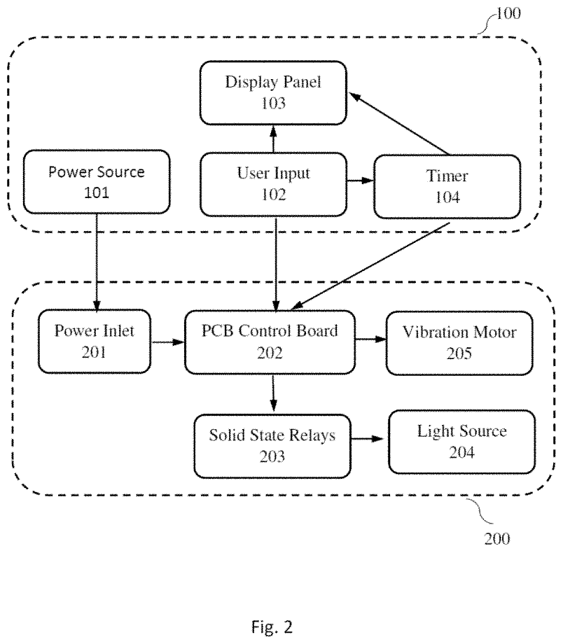

FIG. 2 further illustrates the internal components of the lipolysis apparatus. The control unit (100) houses a power source (101) which provides power to the treatment applicator (200) via the connection cable (300) and a power inlet (103). The operator input (102) component of the control unit (100) is connected to the PCB (printed circuit board) control board (202) of the treatment applicator (200) via the connection cable (300) as well. The operator input (102) also sets a timer (104). The display panel (103) displays system status, operator input, and the timer information. The PCB control board (202) is electronically connected to one or more solid state relays (203) that control that switch on and off the light sources (204). The PCB control board (202) is also electronically connected to and controls at least one vibration motor (205) that is/are housed in the treatment applicator (200) or attached to the treatment applicator (200).

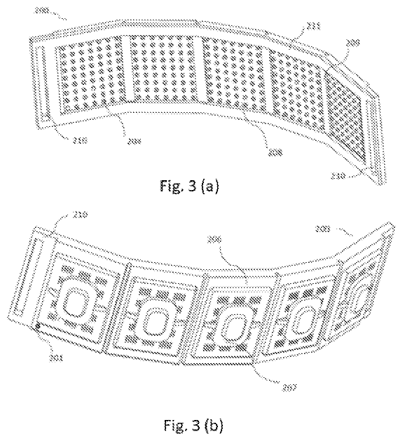

FIG. 3 shows the prospective front and back views of an embodiment of the treatment applicator (200). In this embodiment, the treatment applicator (200) comprises of multiple treatment panels (206). Each treatment panel (206) houses a PCB control board (202), a plurality of solid state relays (203), a plurality of light sources (204), such as light emitting diodes (LEDs), and a vibration motor (205) located in the motor housing (207) at the back of the treatment panel (206). The front of the treatment panel (206) comprises a transparent window (208) that allows light to pass through to reach the skin treatment area. The plurality of treatment panels (206) are arranged in an applicator frame (209) to form a belt-shaped applicator. Each end of the applicator frame comprises a strap loop (210). The applicator frame (209) also comprises a flexible contact frame (211) that is in touch with the patent skin during treatment. The flexible contact frame (211) is made of flexible materials, such as suitable synthetic polymer, and may be shaped to desirable contours to accommodate various sizes of the treatment area.

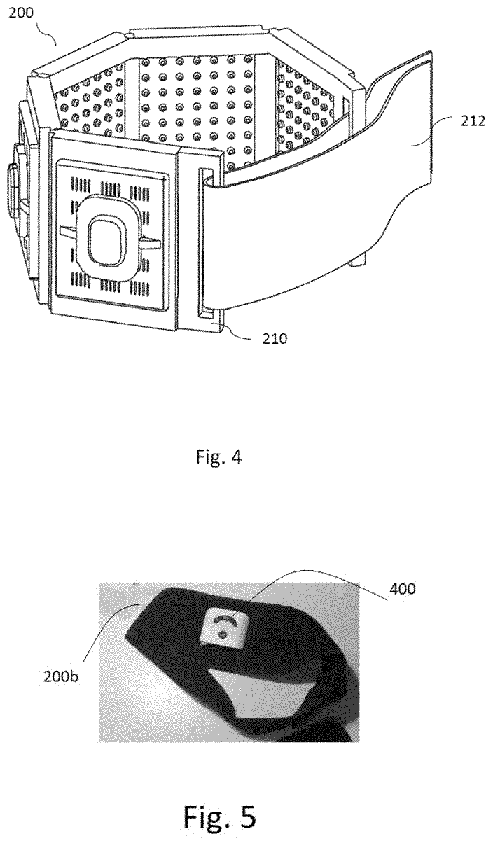

FIG. 4 shows the treatment applicator (200) configured in a bent contour to fit a treatment area, such as an abdomen and waist area. A section of strap (212) is fit through the strap loops (210) to fix the treatment applicator to the user's body. Velcro patches, clips, or other mechanisms can be used for fastening the strap.

Embodiments of the low powered light source (204) can be a light emitting diodes (LEDs) or a low powered laser (s). In some embodiments, their output power ranges from 10 MW to 50 MW. In other embodiments, they operate in the red wavelength range of 620 nm to 629 nm or in the range from 661 nm to 669 nm. There may be an additional heat dissipating plate located in the treatment panel (206) for dissipating heat generated from the light sources. The transparent windows (208) can be embodied as lenses to focus light to desirable intensity on the treatment area. The lipolysis device may be operated in a rhythm mode in which the vibration is repeatedly ran through a multitude of programs at predetermined intervals. The duration and the mode of operation can be setup by the operator using the operator input buttons (102). The applicator frame (209) may have grips formed at appropriate places on one or both sides thereof.

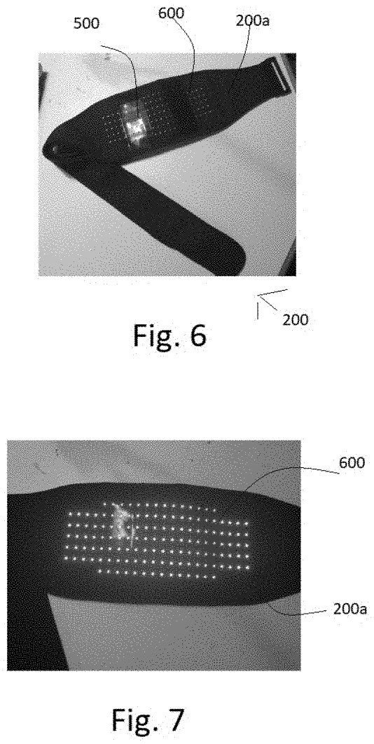

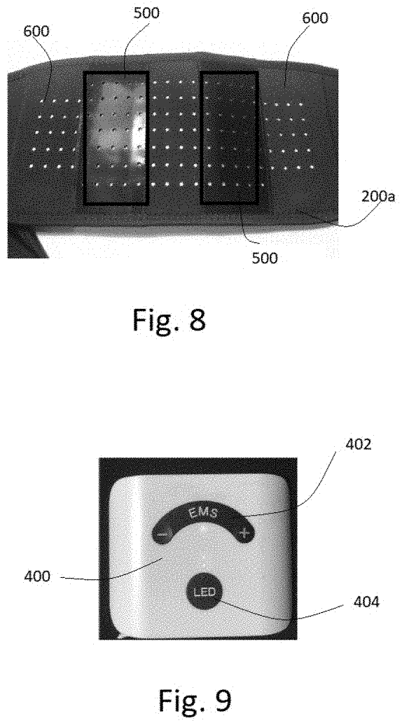

As seen in FIGS. 5-9, in a further embodiment of the present invention, the lipolysis apparatus 200 eliminates the vibration motor 205 and its controls 100 and substitutes it with a rechargeable battery powered dual control unit 400 that operatively connects to the low powered light source 600 and to at least one Electronic Muscle Stimulator 500 that is attached to the treatment applicator 200. The treatment applicator 200 has an Inner surface 200a and an outer surface 200b. The at least one Electronic Muscle Stimulator 500 will be placed on treatment applicator 200 and the low powered light source 600 are defined on the inner surface 200a of the treatment applicator 200. The low power light source 600 can be either a plurality of low power lasers or a plurality of light emitting diodes. The low powered light source is more fully described above as reference numeral 204.

The dual control unit 400 defines a low power light source operator input control 402 and an Electronic Muscle Stimulator operator input control 404. The dual control unit 400 is attached to the treatment applicator 200 on its outer surface 200b.

The Electronic Muscle Stimulator 500 will deliver a pulse rate between 1 Hz to 500 Hz to irradiated areas. The irradiated areas will develop muscle tone while adipose is removed.

In another embodiment of the present invention, the low power light source shall emit a wavelength in the range from about 620 nm to about 629 nm to a patient's treatment area. In still another embodiment of the present invention, the low power light source shall emit a wavelength in the range from about 661 nm to about 900 nm to a patient's treatment area.

The foregoing description and accompanying drawings illustrate the principles, preferred or example embodiments, and modes of assembly and operation, of the invention; however, the Invention is not, and shall not be construed as being exclusive or limited to the specific or particular embodiments set forth hereinabove.

What is claimed is:

1. A lipolysis apparatus for fat removal, body shaping, body contouring and muscle toning, the lipolysis apparatus comprises:
a treatment applicator, wherein the said treatment applicator comprises at least one low power light source that will emit a wavelength to a patient's treatment area;
at least one Electronic Muscle Stimulator that is attached to the treatment applicator; and
a dual control unit that powers the treatment applicator and the at least one Electronic Muscle Stimulator.

2. The lipolysis apparatus of claim 1, wherein the power output of the at least one low power light source is in the range that is between from about 10 MW to about 50 MW.

3. The lipolysis apparatus of claim 2, wherein the low power light source is a light emitting diode (LED).

4. The lipolysis apparatus of claim 3, wherein the treatment applicator defines an inner and an outer surface and wherein a plurality of transparent windows or lenses are disposed on the inner surface, and a plurality of LED diodes that are located within the transparent windows or lenses.

5. The lipolysis apparatus of claim 4, further comprises of at least one heat dissipating plate, wherein the at least one heat dissipating plate is mechanically connected to the treatment applicator.

6. The lipolysis apparatus of claim 1, wherein the treatment applicator can be bent to conform to the contours of a treatment area.

7. The lipolysis apparatus of claim 6, wherein the treatment applicator comprises:
a flexible frame that can be bent up to 360 degrees; and
a plurality of treatment panels, wherein each treatment panel comprises of a plurality of LED light sources, a transparent window that allows light to pass through to reach the treatment area.

8. The lipolysis apparatus of claim 7, further comprises a fastener that will attach to a patient's treatment area.

9. The lipolysis apparatus of claim 8, wherein the fastener comprises of at least one strap loop on the flexible frame and at least one strap that can be fastened to the patient.

10. The lipolysis apparatus of claim 1, wherein the dual control unit has a low power light source operator input control and an Electronic Muscle Stimulator operator input control.

11. The lipolysis apparatus of claim 1, wherein the low power light source is a low power laser.

12. The lipolysis apparatus of claim 1, wherein the low power light source emits a wavelength in the range from about 620 nm to about 629 nm.

13. The lipolysis apparatus of claim 12, wherein the EMS unit has a pulse rate that is between 1 Hz. to 500 Hz.

14. The lipolysis apparatus of claim 13, wherein the low power light source is a red light.

15. The lipolysis apparatus of claim 1, wherein the low power light source emits a wavelength in the range from about 661 nm to about 900 nm.

16. The lipolysis apparatus of claim 15, wherein the EMS unit has a pulse rate that is between 1 Hz. to 500 Hz.

17. The lipolysis apparatus of claim 16, wherein the low power light source is a red light.

18. The lipolysis apparatus of claim 1, wherein the low power light source is either a low power laser or an LED light source.
